(19) United States
(12) Patent Application Publication
(10) Pub. No.: US 2007/0243268 A1
(43) Pub. Date: Oct. 18, 2007

(54) Compositions for regulating intestinal disorders and methods of use thereof

(76) Inventor: Russell M. Jaffe, Sterling, VA (US)

Correspondence Address: COOLEY GODWARD KRONISH LLP;ATTN: Patent Group, Suite 500, 1200 - 19th Street, NW, WASHINGTON, DC, 20036-2402, US

(21) Appl. No.: 11/593,638

(22) Filed: Nov. 7, 2006

Related U.S. Application Data
(60) Provisional application No. 60/733,784, filed on Nov. 7, 2005.

(51) Int. Cl. A61K 33/04; A61K 31/70; A61K 36/48; A61P 3/04; A61P 9/10; A61P 3/10; A61P 1/00; A61K 31/702

(52) U.S. Cl. 424/702; 424/757; 514/25; 514/54

(57) ABSTRACT

The invention relates to compositions comprising one or more prebiotics (e.g., one or more dietary fibers) in combination with selenium compounds, flavonoids and/or flavonols, and phosphatides. The invention also relates to methods of regulating disorders, specifically disorders of the intestinal tract and related disorders, by administering a composition of the invention.

### CROSS REFERENCES TO RELATED APPLICATIONS

The present application claims priority to U.S. Provisional Application 60/733,784, filed Nov. 7, 2005, which is herein incorporated by reference in its entirety for all purposes.

### FIELD OF THE INVENTION

The invention relates to compositions comprising one or more prebiotics, e.g., one or more dietary fibers, in combination with selenium, proanthocyanidins, and phosphatides. The invention also relates to methods of regulating disorders, specifically disorders of the intestinal tract and related disorders, by administering a composition of the invention.

### BACKGROUND OF THE INVENTION

Many of the major problems in human health revolve around which dietary components are truly essential for animal and human health and which components are merely hyped by various companies to sell a product. A related problem is that of the accuracy of information regarding the appropriateness of a given food, nutrient or nutraceutical for a given individual. Certainly the “one size fits all” scenario is untrue when it comes to pharmaceuticals and nutrition. The present invention is concerned with providing a composition that can be beneficial to humans and animals with few, if any, dangers or drawbacks.

Excessive compaction and hardness of digestive residues resulting from constipation and potential accumulations of toxins in the bowel, which toxins may ultimately be absorbed into the blood stream is a serious problem. Further, there is abundant evidence that constipation may lead to a myriad of medical problems related to the gastrointestinal track including colon cancer, possibly as a result of prolonged contact between cells of the colon and toxin laden feces.

Unquestionably as a society, we are suffering from a deplorable lack of dietary fiber. We are constantly warned by the medical profession and other experts that this lack of fiber can and does kill. Our diets are replete with “empty” calories—refined foods loaded with fats and sugars—and contain few whole foods. When it comes to fiber many believe that a daily bowl of cereal is adequate. Our supermarkets and pantries are stuffed with brightly packaged, overly refined, prepared foods that are usually fiber-free or very low in fiber. The presence or absence of dietary fiber greatly influences one's ability to expel solid wastes. It has been estimated that about one in 19 individuals in our society has a health condition that requires special attention. In many cases this makes the need for adequate fiber and water, even more important to these individuals. Due to modern medicine's success in combating contagious disease, and with a better understanding of aging and our ability to medically address the aging process, we are living longer.

Fiber or “roughage” is a component of food that remains undigested as it passes through the gastrointestinal system. The vast majority of dietary fiber consists of polysaccharides of plant origin. The most obvious fiber is the cellulosic wall that surrounds plant cells. Many of these cells are actually called “fibers,” hence the name “fiber” for this dietary component. However, there are actually two forms of fiber: insoluble fiber—the classic cellulosic material, and soluble fiber—water soluble polysaccharides that are not digested by human or carnivore digestive systems. Both types of fiber bind considerable water and, thus, have a softening effect on the stool. However, soluble fiber may, depending on the precise polysaccharides involved, be metabolized or partially metabolized directly by bacteria in the colon. Both types of fibers tend to increase motility within the gastrointestinal tract thus speeding transit time of wastes and lowering the risk of acute and chronic medical problems. Like water, fiber is essential for human health and is not metabolized by humans.

Fiber was removed from food products because in many cases it made the foods coarse, unpalatable or difficult to process. Adding insoluble bran or other similar fiber to foods may provide more roughage but can also degrade the favorable properties of the foods. For example, cakes or pastries made from flours high in insoluble fiber may have inferior taste and texture. Excess insoluble fiber may upset the digestion and lead to a number of digestive problems. On the other hand, soluble fiber is generally well tolerated, often improves the texture or other physical characteristics of the food product and is generally innocuous. Consequently, there are a growing number of food products, ranging from baked goods to “shake-like” beverages, contain added fiber in the form of soluble fiber.

It has been discovered that dietary fiber appears to moderate the rate at which sugars and fats are absorbed from the intestine. In the case of simple sugars, slowed absorption translates to a more gradual rise in blood sugar following eating. This is important in the managing of diabetes and may also help prevent adult onset diabetes. In the case of fats, the fiber seems to help prevent damaging levels of cholesterol in the blood. This seems to be due to a binding of bile salts and cholesterol to the fiber so that these materials are excreted with the feces rather than being absorbed or reabsorbed. Studies show adequate fiber clearly lowers the risk of heart disease and tends to bind toxins, including toxic metals, allowing them to exit safely from the digestive system.

Despite the current existence of dietary fiber products, there is a need for a composition that is easy to take and provides a source of dietary fiber that helps to regulate disorders associated with the intestine.

Selenium is an essential component of at least 11 selenoenzymes or selenoproteins. There are two major families of selenoenzymes-glutathione peroxidases and deiodinases. The metabolic function of the glutathione peroxidases is to convert oxidized fat (lipid hydroperoxides), which is generated as the result of normal metabolism and contributes to heart disease and stroke, to less harmful compounds. This activity is similar to the antioxidant activity of vitamin E. The deiodinase enzymes regulate the metabolism of thyroid hormones. The recently discovered selenoenzyme thioredoxin reductase has been suggested to play a role in vitamin C metabolism.

A human disease known to be caused by selenium deficiency and found in various regions of China is Keshan disease, a cardiomyopathy (i.e., disease of the heart muscle) in children.

Fingernail brittleness and hair loss were used by the Chinese scientists as the main criteria for chronic selenium toxicity, or selenosis, which occurs at an intake of about 5 mg (5,000 mcg) of selenium daily. Adverse effects were observed at daily dietary selenium intakes between about 600 and 1,600 mcg. The maximum safe dietary selenium intake was calculated to be about 800 mcg/day, but may be as low as 600 mcg in some individuals. The Chinese scientists suggested a level of about 40 mcg daily as the minimum requirement, which is similar to the new RDA of 55 mcg/day. This RDA established by the Panel is based on the saturation of plasma glutathione peroxidase. An intake of less than 11 mcg daily of selenium will definitely put one at risk of deficiency.

Early epidemiological studies suggested a possible inverse relationship between selenium intake in humans and the incidence of certain cancers. More than 100 relevant experiments with animals exposed to various chemical and viral carcinogens have been carried out. The majority of these studies showed anticancer effects of selenium. Three human trials on selenium and cancer have been completed, and all of them showed positive results. In one trial, the addition of selenium to table salt significantly reduced the incidence of liver cancer in a Chinese population. After 5 years of supplementation with selenium, vitamin E, and beta-carotene, the incidence of stomach and esophageal cancer in another Chinese population was significantly reduced. However, it is not clear which supplement was mainly responsible for this effect. A study in the U.S. showed that 970 men supplemented with 200 mcg of selenium daily (as selenium-enriched yeast) for 4.5 years had a 63% reduction in the incidence of prostate cancer, as well as a significantly reduced incidence of colorectal, lung, and total cancers. These supplementation studies are consistent with a recent study showing one-half to two-thirds reduction in the risk of prostate cancer among men with the highest selenium status, as assessed by toenail levels of selenium that indicate long-term selenium intake. Overall, the evidence that selenium can lower the risk of prostate and possibly other human cancers was considered very promising, but it concluded that there is currently no proof for an anticancer effect of selenium.

It is estimated that Americans consume about 100 mcg/day of dietary selenium. In the aforementioned prostate cancer study, subjects were given 200 mcg supplements daily, which boosted their estimated daily intake to about 300 mcg. To prevent selenium deficiency symptoms, a daily intake of 55 mcg is required. For maximal protection against certain cancers, a total daily intake of 200-300 mcg is probably necessary.

Accordingly, there is also a need for a composition that is easy to take and provides a source of selenium.

Proanthocyanidins are known to show a variety of biological activities, including antitumor, anti-inflammatory, anti-aging, antioxidant, antiallergy, and antibacterial properties. It is believed that the antioxidant effects of proanthocyanidins can reduce incidence of coronary artery disease and play a role in the stabilization of collagen and maintenance of elastin in connective tissue.

Accordingly, there is a need for a composition which is easy to take and provides a source of proanthocyanidins.

Phosphatides (also known as phospholipids) are a natural constituent of cells in the human body, and are important in supporting a healthy nervous system. Phosphatides are found in the myelin sheath, which is a fatty protective covering for the nerves. I and

Accordingly, there is a need for a composition which is easy to take and provides a source of phosphatides.

### SUMMARY OF THE INVENTION

In one embodiment, the present invention is directed to a composition comprising:

one or more selenium compounds;

one or more prebiotics;

one or more phosphatides or salts thereof;

one or more flavonols and/or flavonoids;

optionally one or more additives.

In another embodiment, the present invention is directed to a method for regulating or treating a condition or disease in a mammal comprising administering to a mammal and effective amount of a composition comprising:

one or more selenium compounds;

one or more prebiotics;

one or more phosphatides or salts thereof;

one or more flavonols and/or flavonoids; and

optionally one or more additional additives.

In yet another embodiment, the present invention is directed to a kit for regulating a condition in a mammal comprising:

a container comprising:

i. one or more selenium compounds;

ii. one or more prebiotics;

iii. one or more phosphatides or salts thereof;

iv. one or more flavonols and/or flavonoids;

v. optionally one or more additional additives; and

vi. instructions for use.

### DETAILED DESCRIPTION OF THE INVENTION

In its many embodiments, the present invention is directed to compositions and kits comprising the composition of the invention, and methods of regulating or treating a condition or disease in a mammal with the composition of the invention, as described herein.

Selenium Compounds

The compositions of the invention comprise selenium compounds. Any pharmaceutically or nutritionally acceptable form of selenium is suitable for use in the compositions of the present invention. Non-limiting examples of suitable forms of selenium include, e.g. selenium, selenium salts such as sodium selenite and sodium selenate, selenomethionine, selenocysteine, selenium proteinates, selenium amino acid chelates, etc., or mixtures thereof. In one embodiment, the compositions of the present invention comprise selenium in the form of selenomethionine.

The selenium or salts thereof of the invention are present in a unit dose of the composition in an amount of about 10 mcg (micrograms) to about 500 mcg, or about 20 mcg to about 400 mcg, or about 50 mcg to about 300 mcg, or about 100 mcg to about 200 mcg, or about 100 mcg to about 150 mcg, including about 10 mcg, about 25 mcg, about 50 mcg, about 75 mcg, about 100 mcg, about 125 mcg, about 150 mcg, about 175 mcg, about 200 mcg, about 225 mcg, about 250 mcg, about 275 mcg, 300 mcg, about 325 mcg, 350 mcg, about 375 mcg, about 400 mcg, about 425 mcg, about 450 mcg, about 475 mcg, or about 500 mcg, inclusive of all ranges and subranges therebetween.

The term “unit dose” means a unitary, i.e. a single dose which is capable of being administered to a patient comprising either the active ingredients as such (e.g., a selenium compound, one or more prebiotics, one or more phosphatides or salts thereof, and one or more proanthocyanidins) or a mixture of the active ingredients with optional additional additives.

In one embodiment, a unit dose of the compositions of the present invention comprises about 125 mcg of selenomethionine.

Alternatively, the concentration of selenium or salts thereof in the compositions of the present invention can be expressed in units of ppm (i.e., “parts per million”). For example, a selenium or salts thereof in the compositions of the present invention are present in a concentration range of about 5 ppm to about 50 ppm, about 10 ppm to about 25 ppm, including about 5 ppm, about 10 ppm, about 15 ppm, about 18 ppm, about 20 ppm, about 25 ppm, about 30 ppm, about 35 ppm, about 40 ppm, about 45 ppm, about 50 ppm, inclusive of all ranges and subranges therebetween (wherein the concentration of selenium or salts thereof is relative to the combined amounts of selenium or salts thereof, one or more prebiotics, one or more proanthocyanidins, and one or more phosphatides or salts thereof). In one embodiment, the compositions of the present invention comprise about 18 ppm of selenomethionine.

Prebiotics

The compositions of the invention also comprise one or more prebiotics. As used herein, the term “prebiotic” refers to indigestible carbohydrates. These prebiotics stimulate the growth and activity of beneficial bacteria of the intestinal flora. The compositions of the invention comprising one or more prebiotics therefore regulate inhibition of possible pathogenic bacteria, have a positive influence on the activity of the immune system; help recovery of the intestinal flora after treatment with antibiotics; aid in production of digestive enzymes; and inhibit viruses (e.g., rota viruses)

The prebiotic said the invention include, for example, dietary fiber. Dietary fibers are the indigestible portion of plant foods that move food through the digestive system and absorb water. Chemically, dietary fiber consists of non-starch polysaccharides and several other plant components such as cellulose, lignin, waxes, chitins, pectins, β-glucans, insulin and oligosaccharides. As used herein, the term “dietary fiber” includes any water-soluble or water-insoluble carbohydrate polymer provided no human enzymes or bacteria common in the human gut are capable of metabolizing, e.g., hydrolyzing these polysaccharides into simple sugars so that they can continue to provide a “bulking” effect.

The prebiotics of the invention include, for example, native dietary fiber. Native dietary fiber includes dietary fiber, as described herein, which is essentially unchanged or chemically identical to its natural state in the plant source from which it was derived. Native dietary fiber can include dietary fiber present in plant material which has been dried and/or reduced to a particulate form. For example, native dietary fiber can be obtained from plant material by milling or extraction under “mild” conditions, e.g. under conditions of low temperature, low-pressure, and/or low shear.

The prebiotics of the present invention also include soluble (i.e., water-soluble) dietary fiber, including any type of soluble fiber which is metabolized by and promotes the growth of beneficial bacteria. This generally has a positive effect as the beneficial bacterial can also tend to lubricate the stool and/or prevent the growth of other bacteria that may release toxins (Leon Prosky, J. of AOAC Int'l. 82:223-35(1999), incorporated herein by reference).

Soluble fiber can also improve the characteristics of fiber-poor refined foods, and restore the benefits of fiber to a highly refined diet. Soluble fiber suitable for the present compositions can be derived from a wide range of plant sources. Non-limiting examples of soluble fiber from plant sources includes water-soluble plant pectins and pectic materials, galactomannans, arabanogalactans and water-soluble hemicelulose; plant “mucilages,” gums, and soluble polysaccharides found in grains, seeds, or stems such as psyllium, guar, oat (beta glucans), astragalus (gum traganth), gum ghatti, gum karaya (Sterculia gum), and gum acacia; algal polysaccharides such as agar or carrageenan; other indigestible carbohydrates, such as dextrans, maltodextrins or dextrins, produced by chemical or enzymatic digestion (e.g., partial hydrolysis) of starch, gums and other carbohydrate polymers; soluble cellulosic ethers and other derivatives such as carboxymethyl cellulose. Other suitable soluble fibers include, e.g., indigestible carbohydrate polymers artificially prepared using bacterial enzymes and non-digestible storage carbohydrates such as insulin. In addition, suitable soluble fibers includes those which are commercially available.

The prebiotics of the present invention can also include insoluble (i.e., water-insoluble) fiber. Insoluble fiber includes indigestible portions of plants, as described herein which are not readily soluble in water. Suitable insoluble fibers include insoluble fibers derived from whole wheat, wheat and corn bran, flax seed lignin and vegetables such as carrots, celery, green beans and potato skins.

It will be recognized that the prebiotics of the present invention can include native dietary fiber, processed dietary fiber, insoluble dietary fiber, soluble dietary fiber, and combinations thereof. For example, the compositions of the present invention can include a combination of soluble and insoluble dietary fiber. In one embodiment, the compositions of the present invention comprise a prebiotic which is a mixture of gum acacia, glucomannan, oat fiber, and dextran. In another embodiment, the prebiotic is an native dietary fiber, for example, the dietary fiber is gum acacia, glucomannan, oat fiber, or one or more fructooligosaccharides or combinations thereof.

It has been discovered that the compositions of the present invention comprising combinations of prebiotics, for example, one or more native dietary fibers, appear to moderate the rate at which sugars and fats are absorbed from the intestine. In regard to simple sugars, the slowed absorption provided by the prebiotic results in a more gradual rise in blood sugar following eating. This is important in the management of diabetes and may also help to prevent adult onset diabetes. In regard to fats, the prebiotics seem to help prevent damaging levels of cholesterol in the blood by binding bile salts and cholesterol to the fiber. Consequently, bile salts and cholesterol are excreted with the feces rather than absorbed or reabsorbed by the gastrointestinal tract. In addition, studies show that adequate levels of dietary fiber clearly lower the risk of heart disease and tend to bind toxins, including toxic metals, thereby allowing them to be excreted safely from the digestive system.

The prebiotics of the present invention, for example one or more dietary fibers, are present in a unit dose of the compositions of the present invention in an amount of from about 100 mg to about 10 g, about 200 mg to about 8 g, about 500 mg to about 7 g, including about 100 mg, about 200 mg, about 300 mg, about 400 mg, about 500 mg, about 600 mg, about 700 mg, about 800 mg, about 900 mg, about 1 g, about 1.5 g, about 2 g, about 2.5 g, about 3 g, about 3.5 g, about 4 g, about 4.5 g, about 5 g, about 5.5 g, about 6 g, about 6.5 g, about 7 g, about 7.5 g, about 8 g, about 8.5 g, about 9 g, about 9.5 g, about 10 g, inclusive of all ranges and subranges therebetween.

In one embodiment, a unit dose of the compositions of the present invention comprise about 7 g of prebiotic. In yet another embodiment, a unit dose of the compositions of the present invention comprise about 3.5 g gum acacia, 2 g glucomannan, 1 g of fiber, and about 500 mg of the dextran.

Alternatively, the concentration of prebiotics in the compositions of the present invention can be expressed in units of wt. % (i.e., “weight percent”, which is the weight of prebiotic divided by the total weight of the composition, multiplied by 100%). For example, a suitable concentration of prebiotic in the compositions of the present invention can range from about 50 to about 99.5 wt. %, from about 60 to about 99.5 wt. %, from about 70 to about 99.5 wt. %, from about 80 to about 99.5 wt. %, including about 50 wt. %, about 60 wt. %, about 70 wt. %, about 80 wt. %, about 90 wt. %, about 95 wt. %, about 96 wt. %, about 97 wt. %, about 98 wt. %, about 99 wt. %, about 99.1 wt. %, about 99.2 wt. %, about 99.3 wt. %, about 99.4 wt. %, or about 99.5 wt. %, inclusive of all ranges and subranges therebetween (wherein the concentration of one or more prebiotics is relative to the combined amounts of selenium or salts thereof, one or more prebiotics, one or more proanthocyanidins, and one or more phosphatides or salts thereof). In one embodiment, the concentration of prebiotic is about 99.3 wt. %. In another embodiment, the concentration of prebiotic is about 50 to about 55 wt. % gum acacia, about 25 wt. % to about 30 wt. % glucomannan, about 12 wt. % to about 17 wt. % oat fiber, and about 5 wt. % to about 10 wt. % dextran.

Flavonols and Flavonoids

The term flavonoid refers to a class of plant secondary metabolites based around a phenylbenzopyrone structure and are also commonly referred to by the equivalent term “bioflavonoid”. Flavonoids include e.g., flavones, flavonols, flavanones, flavan-3-ols, isoflavones, anthocyanadins, and proanthocyanidins. Flavones include e.g. luteolin and apigenin. Flavonols include e.g. quercetin, kaempferol, myricetin, isorhamnetin, pachypodol, and rhamnazin. Flavanones include e.g. hesperetin, naringenin, and eriodictyol. Flavan-3-ols include e.g. (+)-catechin, (+)-gallocatechin, (−)-epicatechin, (−)-*epigallocatechin, (−)-epicatechin 3-gallate, (−)-epigallocatechin 3-gallate, theaflavin, theaflavin 3-gallate, theaflavin 3′-gallate, theaflavin 3,3′ digallate, and thearubigins. Isoflavones include e.g. genistein, daidzein, and glycitein. Anthocyanidins include e.g. cyanidin, delphinidin, malvidin, pelargonidin, peonidin, and petunidin.

In one embodiment, the compositions of the present invention comprise one or more flavonoids and/or flavonols.

In another embodiment, the compositions of the present invention comprise one or more proanthocyanidins, e.g. one or more orthoproanthocyanidins. Proanthocyanidins are also called “OPCs” for oligomeric procyanidins or “PCOs” for procyamidolic oligomers. OPCs are found in many woody plants. The two most common sources of purified and isolated proanthocyanidins are extracts of grape seeds (Vitis vinifera) and the white pine (Pinus maritima, P. pinaster), each of which can be utilized in the compositions of the invention.

The proanthocyanidins of the invention include naturally occurring polyphenolic bioflavonoids that are present in extracts of many fruits and vegetables. For example, sources of purified and isolated proanthocyanidins include, but are not limited to, extracts of grapes, apples, barley, persimmons, coconut, cacao, blueberries, strawberries, adzuki beans, chicory, and peanuts. Such plants preferably belong to the genera Vitis, Malus, Hordeum, Diospyros, Cocos, Theobroma, Pinus, Vaccinium, Fragaria, Phaseolus or Arachis. Proanthocyanidins can also be obtained optionally by purification and isolation from fermentation products of suitable extracts, such as wine, apple wine and beer. It will be recognized by the skilled artisan that an extract comprising proanthocyanidins comprises at least one proanthocyanidin, but more typically mixtures of one or more proanthocyanidins.

The flavonoids, e.g. proanthocyanidins suitable for use in the invention are those that have antiviral, antibacterial, anti-inflammatory and/or antiallergic activities, and are also protect against oxidative damage of tissue by free radicals.

In one embodiment, the flavonoids and/or flavonols of the present composition is grape seed proanthocyanidin extract (GSPE). GSPE demonstrates significant antioxidant activity in liver and brain tissue, compared to controls. GSPE decreases chemically-induced DNA damage, lipid peroxidation, and production of oxygen free radicals. It also provides better protection against oxidative damage than the same doses of other antioxidants, including vitamin C, vitamin E succinate, and β-carotene. The proanthocyanidins of the invention are better at scavenging free radicals and preventing oxidative damage to brain and liver tissue than other antioxidants.

Extracts of flavonoids and/or flavonols, e.g., GSPE, can be prepared by conventional methods. For example, a plant material (e.g., grape seeds) is pulverized or finely cut and then extracted using a solvent. As the extraction solvent, one or more hydrophilic or lipophilic solvent can be used alone, sequentially or together in admixture. Such solvents are preferably selected from solvents such as water, alcohols such as ethanol, methanol and isopropanol, ketones such as acetone and methyl ethyl ketone and esters such as methyl acetate and ethyl acetate. The extraction temperature is generally from 0 to 100° C. or from 5 to 50° C. The extraction time is approximately from 1 hour to 10 days, and the amount of the solvent is generally from 1 to 30 times by weight, or from 5 to 10 times by weight, based on the dry material. The extraction step may be carried out by either stirring, or soaking and standing. If needed, the extraction step may be repeated 2 or 3 times. The crude extract can be obtained, for example by removing any insoluble residue (e.g., by filtration or centrifugation), or by squeezing the pulverized or cut material to remove the liquid. The flavonoids and/or flavonols can then be used in the form of the crude extract, or can be further purified, e.g., by evaporation of the extraction solvent(s), or using additional purification steps as described herein.

Additional purification and isolation of the flavonoids and/or flavonols can also be carried out using, for example, methods known in the pharmaceutical arts for purifying active pharmaceutical compounds. Such methods can include, for example, two-phase solvent partition, column chromatography and/or preparative high performance liquid chromatography, alone or in combination. Examples of the two-phase solvent partition include methods in which oil soluble components and pigments are extracted with a hydrophobic solvent such as n-hexane or petroleum ether and removed, and methods in which the extract is partitioned into a solvent such as n-butanol or methyl ethyl ketone and water to recover proanthocyanidins from the solvent phase. Examples of the column chromatography include ion-exchange column chromatography which uses a carrier such as Amberlite IR-120B or Amberlite IRA-402, absorption column chromatography which uses a carrier such as normal phase silica gel, reverse phase silica gel, Diaion HP-20 or Sepabeads SP-207 and gel filtration which uses a carrier such as Sephadex LH-20. Examples of the preparative high performance liquid chromatography include a method which uses a reverse phase column containing a carrier such as octadecyl silica and a method which uses a normal phase column containing a carrier such as silica gel. Again, these methods can be used as desired alone or in combination and repeatedly. By these purification methods, impurities including water-soluble ionic substances such as salts, nonionic substances such as saccharides and polysaccharides, oil contents and pigments can be removed from the crude extract, and the flavonoids and/or flavonols are thereby purified. Also, proanthocyanidin extracted from grape seeds can be obtained by purifying it in accordance with a method described, for example, in Acta Derm. Venereol. (Stockh.), 78, 428 (1998).

The flavonoids and/or flavonols are present in a unit dose of the compositions of the present invention in an amount of from about 10 mg to about 100 mg, from about 20 mg to about 90 mg, or from about 50 mg to about 75 mg, including about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, inclusive of all ranges and subranges therebetween.

In one embodiment, the amount of flavonoids and/or flavonols present in a unit dose of the compositions of the present invention is about 10 mg to about 15 mg. In another embodiment, the amount of flavonoids and/or flavonols present in a unit dose of the compositions of the present invention is about 12.5 mg.

Alternatively, the concentration of flavonoids and/or flavonols present in the compositions of the present invention can be expressed in units of wt. %. For example, the concentration of flavonoids and/or flavonols present in the compositions of the present invention can be about 0.05 wt. % to about 0.20 wt. %, about 0.10 wt. % to about 0.20 wt. %, about 0.15 wt. % to about 0.20 wt. %, including about 0.05 wt. %, about 0.06 wt. %, about 0.07 wt. %, about 0.08 wt. %, about 0.09 wt. %, about 0.10 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, or about 0.20 wt. %, inclusive of all ranges and subranges therebetween (wherein the concentration of one or more flavonoids and/or flavonols is relative to the combined amounts of selenium compounds, prebiotics, flavonoids and/or flavonols, and phosphatides).

In one embodiment, the concentration of flavonoids and/or flavonols present in the compositions of the present invention is about 0.15 wt. % to about 0.20 wt. %. In another embodiment, the concentration of flavonoids and/or flavonols present in the compositions of the present invention is about 0.17 wt. % to about 0.19 wt. %. In yet another embodiment, the concentration of flavonoids and/or flavonols present in the compositions of the present invention is about 0.18 wt. %.

Phosphatides

The phosphatides suitable for use in the compositions of the invention include any pharmaceutically acceptable phospholipid, including but not limited to, phosphatidyl-choline, phosphatidyl-ethanolamine, phosphatidyl-inositol and mixtures thereof. The term “phosphatides” as used herein also includes “lecithin,” which is the commercial or popular name for a mixture of naturally occurring phosphatides or phospholipids.

The one or more phosphatides of the invention are present in a unit dose of the composition in an amount of from about 10 mg to about 100 mg, from about 20 mg to about 60 mg, or from about 30 mg to about 40 mg, including about 10 mg, about 20 mg, about 30 mg, about 40 mg, about 50 mg, about 60 mg, about 70 mg, about 80 mg, about 90 mg, or about 100 mg, including all ranges and subranges therebetween. In one embodiment, the amount of phosphatide in a unit dose of the compositions of the present invention is about 30 mg to about 40 mg. In another embodiment, the amount of phosphatide in a unit dose of the compositions of the present invention is about 35 mg. In yet another embodiment, the amount of phosphatide in a unit dose of the compositions of the present invention is about 36 mg.

In one embodiment, the one or more phosphatides of the composition of the present invention comprises phosphatidyl choline, phosphatidyl ethanolamine, and phosphatidyl inositol.

Alternatively, the concentration of the one or more phosphatides in the compositions of the present invention can be expressed in units of wt. %. For example the concentration of phosphatide can range from about 0.10 wt. % to about 1.00 wt. %, about 0.20 wt. % to about 0.80 wt. %, about 0.40 wt. % to about 0.60 wt. %, including about 0.10 wt. %, about 0.12 wt. %, about 0.14 wt. %, about 0.16 wt. %, about 0.18 wt. %, about 0.20 wt. %, about 0.22 wt. %, about 0.24 wt. %, about 0.26 wt. %, about 0.28 wt. %, about 0.30 wt. %, about 0.32 wt. %, about 0.34 wt. %, about 0.36 wt. %, about 0.38 wt. %, about 0.40 wt. %, about 0.42 wt. %, about 0.44 wt. %, about 0.46 wt. %, about 0.48 wt. %, about 0.50 wt. %, about 0.52 wt. %, about 0.54 wt. %, about 0.56 wt. %, about 0.58 wt. %, about 0.60 wt. %, about 0.62 wt. %, about 0.64 wt. %, about 0.66 wt. %, about 0.68 wt. %, about 0.70 wt. %, about 0.72 wt. %, about 0.74 wt. %, about 0.76 wt. %, about 0.78 wt. %, about 0.80 wt. %, about 0.82 wt. %, about 0.84 wt. %, about 0.86 wt. %, about 0.88 wt. %, about 0.90 wt. %, about 0.92 wt. %, about 0.94 wt. %, about 0.96 wt. %, about 0.98 wt. %, about 1.00 wt. %, inclusive of all ranges and subranges therebetween (wherein the concentration of one or more phosphatides or salts thereof is relative to the combined amounts of selenium compounds, prebiotics, flavonoids and/or flavonols, and phosphatides).

Additional Additives

The compositions of the present invention can optionally include additional additives. The term “optionally” in regard to the additional additives means that no additional additives are present, or that one or more additional additives are be present. That is, the compositions of the present invention can consist only of one or more selenium compounds, one or more prebiotics, one or more flavonoids and/or flavonols, and one or more phosphatides, or alternatively, in addition to the selenium compound, one or more prebiotics, one or more flavonoids and/or flavonols, and one or more phosphatides, the compositions of the present invention can also include additional additives such as a diluent, a filler, adjuvant, excipient, or carrier.

Non-limiting examples of suitable additional additives include, for example liquids, such as water (e.g., saline) and oils, including those of petroleum, animal, vegetable or synthetic origin, such as peanut oil, soybean oil, mineral oil, sesame oil and the like. In addition, additional additives can include auxiliary, stabilizing, thickening, lubricating and coloring agents; excipients such as starch (or starch paste), mannitol, glucose, lactose, sucrose, gelatin, malt, rice, flour, chalk, silica gel (or colloidal silica), sodium stearate, glycerol monostearate, magnesium stearate, talc, sodium chloride, dried skim milk, glycerol, propylene, glycol, water, ethanol, urea, keratin, cellulose, magnesium carbonate, and the like. Also, the compositions of the present invention can include, for example, sweetening agents such as fructose, aspartame or saccharin (sodium saccharine); flavoring agents such as peppermint, oil of wintergreen, or cherry; coloring agents; and preserving agents, to provide a pharmaceutically palatable preparation. When the compositions of the present invention are intended for human consumption, the additional additives are of pharmaceutical grade.

Moreover, when the compositions of the present invention are prepared in the form of a tablet or pill, the compositions may be coated to delay disintegration and absorption in the gastrointestinal tract (e.g., with an enteric coating), thereby providing a sustained action over an extended period of time. Selectively permeable membranes surrounding an osmotically active driving compound are also suitable for orally administered compounds of the invention. (For these later forms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations.) A time delay material such as glycerol monostearate or glycerol stearate may also be used. The present compositions, if desired, can also contain minor amounts of wetting or emulsifying agents, or pH buffering agents.

Compositions of the Invention

The compositions of the inventions are useful in regulating numerous disorders including, but not limited to, colon cancer, heart disease, cerebral apoplexy, appendicitis, and diabetes. The compositions of the invention are also useful in regulating constipation and digestive problems, or disorders linked to constipation such as intestinal toxemia, hemorrhoids, irritable bowel syndrome (IBS), colitis, diverticulitis, varicocele, and cholelithiasis (gall stones). In addition, The compositions of the invention can perform various useful physiological functions including reduction of serum cholesterol, limitation of insulin secretion, and acceleration of bowel evacuation. Thus, the compositions of the present invention are also useful for treating or regulating atherosclerosis, high blood pressure, diabetes, and hypoglycemia. The compositions of the present invention are also useful in regulating obesity and stress

The combination of the components of the invention can act in a synergistic way and do not exhibit the effects when administered separately.

Due to the activity of the compositions of the invention, the compositions are advantageously useful in veterinary and human medicine. The invention provides methods of regulating disorders by administration to a patient of an effective amount of a composition of the invention. The patient is a mammal, including, but not limited, cat, dog or a human. In one embodiment, the patient is a human.

Various delivery systems are known, e.g., encapsulation in liposomes, microparticles, microcapsules, capsules, etc., and can be used to administer a compound of the invention. The mode of administration is left to the discretion of the practitioner, and will depend in-part upon the site of the medical condition. In most instances, administration will result in the release of at least some of the compounds of the invention into the bloodstream (e.g., selenium).

In another embodiment, the compounds of the invention can be delivered in a vesicle, for example a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the compounds of the invention can be delivered in a controlled release system. In one embodiment, a pump may be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target of the compounds of the invention, e.g., the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

In one embodiment, the compositions of the present invention are administered orally. Any conventional oral form suitable for use with the compositions of the present invention. For example, the compositions of the present invention can take the form of tablets, pills, lozenges, pellets, capsules, capsules containing liquids, powders, granules, sustained-release formulations, suppositories, emulsions, aerosols, sprays, suspensions (oily or aqueous), solutions, syrups, elixirs or any other form suitable for use. Other examples of suitable pharmaceutical vehicles are described in “Remington's Pharmaceutical Sciences” by E. W. Martin. In one embodiment, the compositions of the present invention are in the form of a capsule (see e.g., U.S. Pat. No. 5,698,155). In another embodiment, the compositions of the present invention are in the form of a powder. In yet another embodiment, the compositions of the present invention are in the form of an aqueous suspension or solution of a powder. In still another embodiment, the compositions of the present invention can be administered with water or juice, up to five times per day. Compounds and compositions of the invention for oral delivery can also be formulated in foods and food mixes.

In another embodiment, the compounds of the invention are formulated in accordance with routine procedures as a nutraceutical composition adapted for oral administration to human beings. In another embodiment, the compositions of the invention be administered orally. Compositions for oral delivery may be in the form of pills, tablets, lozenges, aqueous or oily suspensions, granules, powders, emulsions, capsules, syrups, or elixirs, for example. Orally administered compositions may contain one or more additional additives as described herein.

Alternatively, the compositions of the invention may be administered by other routes, including, but not limited to topical, dermal, transdermal, rectal, or slow release formulations. The compositions of the invention may be administered by any convenient route, for example, orally, topically, by absorption through epithelial or mucocutaneous linings (e.g., oral mucosa, rectal and intestinal mucosa, etc.) and may be administered together with another biologically active agent.

Administration can be systemic or local. In certain embodiments, more than one composition of the invention can be administered to a patient. Methods of administration include, but are not limited to intranasal, oral, sublingual, intranasal, intravaginal, transdermal, rectally, by inhalation, or topically, for example, to the ears, nose, eyes, scalp, or skin. The mode of administration is left to the discretion of the practitioner, and will depend in-part upon the site of the condition. In most instances, administration will result in the release of the composition of the invention for maximum uptake by a cell.

In specific embodiments, it may be desirable to administer one or more compositions of the invention locally to the area in need of treatment. This may be achieved, for example, and not by way of limitation, by topical application (e.g., as a cream); by local infusion during surgery (e.g., in conjunction with a wound dressing after surgery); by injection; by means of a catheter; by means of a suppository; or by means of an implant, said implant being of a porous, non-porous, or gelatinous material, including membranes, such as sialastic membranes, or fibers. In one embodiment, administration can be by direct injection at the site (or former site) of an atherosclerotic plaque tissue.

In another embodiment, the composition is prepared in a form suitable for administration directly or indirectly to surface areas of the body for direct application to affected areas. In such situations, the formulation can also include anti-drying agents (e.g., pantethine), penetration enhancers (e.g., dimethyl isosorbide), accelerants (e.g., isopropylmyristate) or other common additives that are known in the industry and used for topical applications (e.g., glycerin, propylene glycol, polyethylene glycols, ethyl alcohol, liposomes, lipids, oils, creams, or emollients). In addition, the compositions of the present invention may include compounds that have a beneficial effect on skin pores, such as retinoic acid (i.e., Retin-A), which removes sebum plugs from pores; antioxidants (e.g., butylated hydroxyanisole); or chelating preservatives (e.g., disodium EDTA).

The addition of various concentrations of the enhancer glycerin have been shown to enhance the penetration of cyclosporin (Nakashima et al., 1996). The use of terpene-based penetration enhancers with aqueous propylene glycol have also shown the capacity to enhance topical delivery rates of 5-fluorouricil (Yamane et al., 1995). 5-fluorouricil, 5-FU, is a model compound for examining the characteristics of hydrophilic compounds in skin permeation studies. Thus, the addition of terpenes in polylene glycol (up to 80%) were able to enhance the flux rate into skin.

Dimethyl isosorbide (DMI) is another penetration enhancer that has shown promise for pharmaceutical formulations. DMI is a water-miscible liquid with a relatively low viscosity (Zia et al., 1991). DMI undergoes complexation with water and polylene glycol but not polyethylene glycol. It is the ability for DMI to complex with water that provides the vehicle with the capacity to enhance the penetration of various steroids. Maximum effects were seen at a DMI:water ratio of 1:2. Evidence in the literature suggests that the effect of pH on DMI is an important consideration when using DMI in various formulations (Brisaert et al., 1996).

The compositions of the present invention may also be administered to a patient via pulmonary administration, (e.g., by use of an inhaler or nebulizer). Thus, the compositions of the present invention may also be formulated with an aerosolizing agent, or via perfusion in a fluorocarbon or synthetic pulmonary surfactant.

In certain embodiments, the compounds of the invention can be formulated as a suppository, with traditional binders and vehicles such as triglycerides.

In another embodiment, the compositions of the invention can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in “Remington's Pharmaceutical Sciences” by E. W. Martin.

The present compositions will contain an effective amount of the components of the invention. “Components of the invention” means the individual “active” components, specifically at least: selenium or salts thereof, one or more prebiotics, one or more phosphatides or salts thereof, and one or more proanthocyanidins. Each of the components of the present invention, as described herein, may be in the form of extracts containing the component as well as other compounds for materials, or in purified form (i.e., the component itself). The term “effective amount” in regard to the components of the invention refers to the amount of each component necessary to provide a clinically useful effect (e.g., preventing, regulating, reducing or ameliorating symptoms associated with a condition such as constipation).

In addition to effective amount of the components of the invention, the compositions of the present invention can also include additional additives such as those described herein. Some additional additives, for example sweetening or flavoring agents, would not typically be considered components of the invention because they do not provide a clinically useful effect in a patient, but instead are intended to improve e.g. the palatability and/or stability of the formulation.

The amount of a component of the invention that will be that amount which is effective in regulating a disorder or condition disclosed herein, and will depend on the nature of the disorder or condition, and can be determined by standard techniques. The precise dose to be employed in the compositions will also depend on the route of administration, and the seriousness of the disease or disorder, and should be decided according to the judgment of the practitioner and each patient's circumstances. In vitro or in vivo assays may optionally be employed to help identify optimal dosage ranges.

Oral compositions can contain 10% to 100% by weight of the components of the invention.

In a particular embodiment, the present invention encompasses a composition comprising:
one or more selenium compounds;
one or more prebiotics;
one or more phosphatides or salts thereof;
one or more flavonoids and/or flavonols; and
optionally one or more additional additives.

In an illustrative embodiment of the composition of the present invention, the prebiotic is gum acacia. In another illustrative embodiment of the composition of the present invention, the prebiotic is glucomannan. In another illustrative embodiment of the composition of the present invention, the prebiotic is oat fiber. In another illustrative embodiment of the composition of the present invention, the prebiotic is one or more fructooligosaccharides.

In a particular embodiment, a unit dose of the compositions of the present invention comprises from about 10 mcg to about 500 mcg of selenium compounds; about 100 mg to about 1000 mg of the prebiotic (e.g., dietary fibers); from about 10 mg to about 100 mg of phosphatides, wherein the phosphatides are selected from the group consisting of phophatidyl-choline, phosphatidyl-ethanolamine, phosphatidyl-inositol and combinations thereof; and from about 1 mg to about 50 mg of one or more flavonoids and/or flavonols, e.g. proanthocyanidins.

In another embodiment of the compositions of the present invention, the selenium compounds are present in an amount of from 15 ppm to 20 ppm, the one or more prebiotics are present in an amount ranging from 98.0 wt. % to 99.5 wt. %, the amount of one or more phosphatides or salts thereof are present in an amount ranging from 0.45 wt. % to 0.55 wt. %, and the amount of one or more flavonoids and/or flavonols, e.g. proanthocyanidins is present in an amount ranging from 0.15 wt. % to 0.20 (wt. %), based on the total weight of the composition.

In another embodiment of the compositions of present invention, the selenium compounds are present in an amount of from 15 ppm to 20 ppm (based on the total weight of the composition). In another embodiment of the compositions of the present invention, the one or more prebiotics are present in an amount ranging from 98.0 wt. % to 99.5 wt % (based on the total weight of the composition). In another embodiment of the compositions of the present invention, the one or more phosphatides or salts thereof are present in an amount ranging from 0.45 wt. % to 0.55 wt. % (based on the total weight of the composition). In another embodiment of the compositions of the present invention, the one or more flavonoids and/or flavonols, e.g. orthoproanthocyanidins are present in an amount ranging from 0.15 wt. % to 0.20 wt. % (based on the total weight of the composition).

In an alternative embodiment, the compositions of the present invention can comprise one or more selenium compounds, one or more prebiotics, and one or more flavinoid and/or flavinols. The amount of selenium compound, based on the total amount of selenium compound, prebiotic, and flavinoid and/or flavinol is about 10-20 parts per million, about 15-20 ppm, or about 17-19 ppm. In one embodiment, the amount of selenium compound, based on the total amount of selenium compound, prebiotic, and flavinoid and/or flavinol is about 18 ppm. The amount of prebiotic, based on the total amount of selenium compound, prebiotic, and flavinoid and/or flavinol is about 97-99.999%, or about 97-99.998%. In one embodiment, the amount of prebiotic, based on the total amount of selenium compound, prebiotic, and flavinoid and/or flavinol is about 99.998%. The amount of flavinoid and/or flavinol, based on the total amount of selenium compound, prebiotic, and flavinoid and/or flavinol is about 0.10-0.30%, about 0.10-0.20%, or about 0.15-0.20%. In one embodiment, the amount of flavinoid and such or flavinol, based on the total amount of selenium compound, prebiotic, and flavinoid and/or flavinol is about 0.18%.

Methods

The present invention encompasses methods of regulating disorders associated with deficiency in dietary fiber including, but not limited to, colon cancer, heart disease, cerebral apoplexy, appendicitis, and diabetes.

The invention also encompasses methods of regulating disorders linked to constipation including, but not limited to, intestinal toxemia, hemorrhoids, irritable bowel syndrome (“IBS”), colitis, diverticulitis, varicocele, and cholelithiasis (gall stones).

The term “regulating disorders” includes preventing or reducing the probability of contracting the disorder, treating the symptoms of the disorder, treating the biological processes or mechanisms underlying the disorder, etc.

A particular embodiment of the invention encompasses a method for regulating a condition in a mammal comprising administering to a mammal an effective amount of a composition comprising:
one or more selenium compounds;
one or more prebiotics;
one or more phosphatides or salts thereof;
one or more flavonoids and/or flavonols; and optionally one or more additional additives.

In an illustrative embodiment of the method of the present invention, the prebiotic is gum acacia. In another illustrative embodiment of the method of the present invention, the prebiotic is glucomannan. In another illustrative embodiment of the method of the present invention, the prebiotic is oat fiber. In another illustrative embodiment of the method of the present invention, the prebiotic is one or more fructooligosaccharides.

In another illustrative embodiment of the method of the present invention, the selenium compounds are present in a unit dose of the compositions of the present invention in an amount of from about 10 mcg to about 500 mcg. In another illustrative embodiment of the method of the present invention, the one or more prebiotic is present in a unit dose of the compositions of the present invention an amount of from about 100 mg to about 1000 mg. In another illustrative embodiment of the method of the present invention, the one or more phosphatides or salts thereof comprise phophatidyl-choline, phosphatidyl-ethanolamine, and/or phosphatidyl-inositol. In another illustrative embodiment of the method of the present invention, the one or more phosphatides or salts thereof are present in a unit dose of the compositions of the present invention in an amount of from about 10 mg to about 100 mg. In another illustrative embodiment of the method of the present invention, the one or more flavonoids and/or flavonols, e.g. orthoproanthocyanidins are present in a unit dose of the compositions of the present invention in an amount of from about 1 mg to about 50 mg.

In another embodiment of the method of present invention, the compositions administered to a mammal comprise selenium compounds are present at a concentration of from 15 ppm to 20 ppm (based on the total weight of the composition). In another embodiment of the method of the present invention, the compositions administered to a mammal comprise one or more prebiotics at concentrations ranging from 98.0 wt. % to 99.5 wt % (based on the total weight of the composition). In another embodiment of the method of the present invention, the compositions administered to a mammal comprise one or more phosphatides or salts thereof at concentrations ranging from 0.45 wt. % to 0.55 wt. % (based on the total weight of the composition). In another embodiment of the method of the present invention, the compositions administered to a mammal comprise one or more flavonoids and/or flavonols, e.g. orthoproanthocyanidins at concentrations ranging from 0.15 wt. % to 0.20 wt. % (based on the total weight of the composition).

In another embodiment of the method of the present invention, the compositions administered to a mammal comprise selenium or salts thereof at concentrations ranging from 15 ppm to 20 ppm, one or more prebiotics at concentrations ranging from 98.0 wt. % to 99.5 wt. %, one or more phosphatides or salts thereof at concentrations ranging from 0.45 wt. % to 0.55 wt. %, and one or more flavonoids and/or flavonols, e.g. proanthocyanidins at concentrations ranging from 0.15 wt. % to 0.20 (wt. %), based on the total weight of the composition.

In another illustrative embodiment of the method of the present invention, the mammal is a human.

In another illustrative embodiment of the method of the present invention the administration is oral.

In another illustrative embodiment of the method of the present invention, the condition being regulated is atherosclerosis, hemorrhoids, constipation, high blood pressure, diabetes, hypoglycemia, digestive problems, obesity, diverticulitis, or stress.

In another illustrative embodiment of the method of the present invention, the method is a method for regulating constipation in a mammal.

The methods and compositions of the present invention are applicable to any mammal. However, the digestive systems of herbivores, for example, ruminates, varies tremendously from that of humans. Accordingly, it is recognized that the compositions and methods of the present invention can be adjusted for the particular needs of a mammal to be treated. For example, the unit dose can be increased or decreased according to the size of the mammal, and the relative amounts of the components of the compositions of the present invention can be adjusted depending upon the particular condition to be treated.

The compositions of the present invention can be administered once daily, or up to five times daily, depending upon the needs of the patient, and the condition to be treated. In addition, the unit dose (typically about 5 to 10 g, e.g., about 7 g) of the compositions of the present invention can be increased or decreased depending upon the needs of the patient. For example, the unit dose could be increased to about 10 g to 20 g, 15 g, to 30 g, etc. as needed, thereby increasing, and a proportionate manner, the amounts of individual components administered to the patient. Thus, for example, if the unit dose of selenium compounds is about 20 mcg when about 7 g of the composition is administered, if the unit dose of the composition was increased to 15 g, the unit dose of selenium compounds would then be about 42.9 mcg. The unit doses of the other components of the composition would likewise increased proportionately.

The composition of the present invention can be administered as a single dose comprising all of the individual components of the composition (e.g., all of the components are present in the form of a powdery mixture). Alternatively, the individual components of the composition of the present invention can be administered, either sequentially or simultaneously, in the form of separate unit doses of each individual component, or as separate unit doses of individual or mixtures of two or more components. For example, the composition of the present invention can be administered, sequentially or simultaneously, as a first unit dose comprising the selenium and prebiotic components, and a second unit dose comprising the phosphatide and proanthocyanidins components. Of course, any combination of unit doses comprising any combination of the component of the present invention could be administered. Furthermore, if the composition of the present invention is administered in the form of two or more separate unit doses, each unit dose could further comprise one or more optional additional additives as disclosed herein.

Kits of the Invention

As described herein, the compositions of the present invention can be administered in any dosage form, for example and oral dosage form. For example, the individual components of the compositions of the present invention can be mixed together into a single dosage form, for example in the form of a powder, capsule, or tablet, whereby consumption of the single dosage form provides simultaneous administration of each of the components of the composition.

Alternatively, the composition of the present invention can comprise separate unit dosage forms of one or more of the components: for example the selenium compounds can be combined with the one or more prebiotics in one dosage form, and the one or more phosphatides or salts thereof and one or more flavonoids and/or flavonols can be combined into a second dosage form. These two dosage forms would then be administered sequentially or simultaneously to obtain the desired effect. Alternatively, each component could be provided in separate unit dosage forms, and administered sequentially or simultaneously to obtain the desired effect.

Whether administered as a single unit dosage form comprising each of the individual components of the composition of the present invention, or in two or more separate unit dosage forms which together provide each of the individual components of the composition of the present invention, the composition of the present invention can be provided in the form of a kit for regulating a condition in a mammal. In one embodiment, a kit comprising the composition of the present invention comprises:

A container comprising:

i. one or more selenium compounds;

ii. one or more prebiotics;

iii. one or more phosphatides or salts thereof;

iv. one or more flavonoids and/or flavonols; and

v. instructions for use,

wherein the one or more selenium compounds, one or more prebiotics, one or more phosphatides or salts thereof, and flavonoids and/or flavonols are optionally each pre-measured into a respective unit of use amount.

Alternatively, in another embodiment, a kit comprising the composition of the present invention comprises:

two or more containers, wherein said two or more containers together comprise:

i. one or more selenium compound;

ii. one or more prebiotics;

iii. one or more phosphatides or salts thereof;

iv. one or more flavonoids in/flavonols; and

v. instructions for use.

In an illustrative embodiment of the kit, the one or more prebiotics are acacia gum, glucomannan, oat fiber, one or more fructooligosaccharides, or combinations thereof. In another illustrative embodiment of the kit, the one or more selenium compounds are present in each unit dose in an amount of from about 10 mcg to about 500 mcg. In another illustrative embodiment of the kit, the one or more prebiotics are present in each unit dose in an amount of from about 100 mg to about 1000 mg. In another illustrative embodiment of the kit, the one or more phosphatides or salts thereof are selected from the group consisting of phophatidyl choline, phosphatidyl ethanolamine, phosphatidyl inositol, and combinations thereof. In another illustrative embodiment of the kit, the one or more phosphatides or salts thereof are present in each unit dose in an amount of from about 10 mg to about 100 mg. In another illustrative embodiment of the kit, the one or more flavonoids and/or flavonols are present in each unit dose in an amount of from about 1 mg to about 50 mg.

In another embodiment of the kit of present invention, the concentration of one or more selenium compounds ranges from 15 ppm to 20 ppm (based on the total weight of the composition). In another embodiment of the kit of the present invention, the concentration of one or more prebiotics ranges from 98.0 wt. % to 99.5 wt % (based on the total weight of the composition). In another embodiment of the kit of the present invention, the concentration of one or more phosphatides or salts thereof ranges from 0.45 wt. % to 0.55 wt. % (based on the total weight of the composition). In another embodiment of the kit of the present invention, the concentration of one or more flavonoids and/or flavonols ranges from 0.15 wt. % to 0.20 wt. % (based on the total weight of the composition).

In another embodiment of the kit of the present invention, the concentration of one or more selenium compounds ranges from 15 ppm to 20 ppm, the concentration of one or more prebiotics ranges from 98.0 wt. % to 99.5 wt. %, the concentration of one or more phosphatides or salts thereof ranges from 0.45 wt. % to 0.55 wt. %, and the concentration of one or more flavonoids and/or flavonols ranges from 0.15 wt. % to 0.20 wt. %, based on the total weight of the composition.

The invention also provides pharmaceutical packs or kits comprising one or more containers filled with one or more compounds of the invention. Optionally associated with such container(s) can be a notice describing the manufacture, use or sale of compositions. In another embodiment, the kit contains more than one compound of the invention.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

### EXAMPLES

A suitable composition of the present invention is shown in Table 1:

| | Amount (per ˜7 g | |
|---|---|---|
| Ingredient | total composition) | |
| Selenium (as 100% I-selenomethionine) | 125 | mcg |
| Gum Acacia (standardized wild-crafted | 3.5 | g |
| gum Arabic) | | |
| Glucomannan (freeze-dried) | 2 | g |
| Oat fiber (14% β-glucan; Oatwell 14 ®) | 1 | g |
| Dextran FOS (short-chained prebiotics) | 500 | mg |
| OPC 85+ ™ (ActiVin IH636TM; | 12.5 | mg |
| proanthocyanidins 92% soluble) | | |
| Phosphatides (includes | 36 | mg |
| phosphatidylcholine, phosphatidyl | | |
| ethanolamine, and phosphatidyl inositol) | | |

The composition of Table 1 is in the form of a powder comprising the indicated components. Approximately 7 g of the composition of Table 1 can be mixed with water or juice and consumed by the patient one or more times daily, as needed.

Various references have been cited herein, each of which is incorporated herein by reference in its entirety for all purposes.

What is claimed is:

1. A composition comprising:
one or more selenium compounds;
one or more prebiotics;
one or more phosphatides or salts thereof;
one or more flavonols and/or flavonoids; and
optionally one or more additional additives.

2. The composition of claim 1, wherein the selenium compound is selected from the group consisting of selenium, sodium selenite, sodium selenate, selenomethionine, selenocysteine, selenium proteinates, selenium amino acid chelates, and mixtures thereof.

3. The composition of claim 1, wherein the one or more prebiotics is native dietary fiber.

4. The composition of claim 1, wherein the one or more prebiotics is selected from the group consisting of acacia gum, glucomannan, oat fiber, fructooligosaccharides, and combinations thereof.

5. The composition of claim 1, wherein the one or more phosphatides are selected from the group consisting of phophatidyl-choline, phosphatidyl-ethanolamine, phosphatidyl-inositol, and combinations thereof.

6. The composition of claim 1, wherein the one or more flavonols and/or flavonoids are selected from the group consisting of grape seed extract and white pine extract.

7. The composition of claim 1, wherein the concentration of one or more selenium compounds ranges from 15 ppm to 20 ppm.

8. The composition of claim 1, wherein the concentration of one or more prebiotics ranges from 98.0 wt. % to 99.5 wt. %.

9. The composition of claim 1, wherein the concentration of one or more phosphatides, or salts thereof, ranges from 0.45 wt. % to 0.55 wt. %.

10. The composition of claim 1, wherein the concentration of one or more flavonols and/or flavonoids ranges from 0.15 wt. % to 0.20 wt. %.

11. The composition of claim 1, comprising:
ppm to 20 ppm of one or more selenium compounds;
98.0 wt. % to 99.5 wt. % of one or more prebiotics;
0.45 wt. % to 0.55 wt. % of one or more phosphatides or salts thereof;
0.15 wt. % to 0.20 wt. % one or more flavonols and/or flavonoids;
optionally one or more additional additives;
wherein the concentrations of one or more selenium compounds, one or more prebiotics, one or more phosphatides or salts thereof, and one or more of the flavonols and/or flavonoids are relative to the total weight of selenium compounds, prebiotics, phosphatides or salts thereof, and flavonols and/or flavonoids.

12. The composition of claim 1, in the form of a powder.

13. The composition of claim 1, in the form of a suspension or solution.

14. The composition of claim 1, further comprising one or more additional additives.

15. A composition comprising:
one or more selenium compounds;
one or more prebiotics;
one or more flavonols and/or flavonoids; and
optionally one or more additional additives.

16. A method for regulating or treating a condition or disease in a mammal comprising administering to a mammal and effective amount of a composition comprising:
one or more selenium compounds;
one or more prebiotics;
one or more phosphatides or salts thereof;
one or more flavonols and/or flavonoids; and
optionally one or more additional additives.

17. The method of claim 16, wherein the condition or disease is selected from the group consisting of atherosclerosis, hemorrhoids, constipation, high blood pressure, diabetes, hypoglycemia, digestive problems, obesity, diverticulitis, and stress.

18. The method of claim 16, wherein the condition or disease is constipation.

19. The method of claim 16, wherein the one or more prebiotics are selected from the group consisting of acacia gum, glucomannan, oat fiber, fructooligosaccharides, and combinations thereof.

20. The method of claim 16, wherein the concentration of one or more selenium compounds ranges from 15 ppm to 20 ppm.

21. The method of claim 16, wherein the concentration of one or more prebiotics ranges from 98.0 wt. % to 99.5 wt. %.

22. The method of claim 16, wherein the one or more phosphatides are selected from the group consisting of phophatidyl-choline, phosphatidyl-ethanolamine, phosphatidyl-inositol, and combinations thereof.

23. The method of claim 16, wherein the concentration of one or more phosphatides, or salts thereof, ranges from 0.45 wt. % to 0.55 wt. %.

24. The method of claim 16, wherein the concentration of one or more flavonols and/or flavonoids ranges from 0.15 wt. % to 0.20 wt. %.

25. The method of claim 16, wherein the mammal is a human.

26. The method of claim 16, wherein said administering is oral administration.

27. A kit for regulating a condition in a mammal comprising:
a container comprising:
i. one or more selenium compounds;
ii. one or more prebiotics;
iii. one or more phosphatides or salts thereof;
iv. one or more flavonols and/or flavonoids;
v. optionally one or more additional additives; and
vi. instructions for use.

28. The kit of claim 27, wherein the one or more selenium compounds, one or more prebiotics, one or more phosphatides or salts thereof, and one or more flavonols and/or flavonoids are each pre-measured into a respective unit of use amount.

29. The kit of claim 27, wherein the one or more prebiotics is selected from the group selected from acacia gum, glucomannan, oat fiber, fructooligosaccharides.

30. The kit of claim 27, wherein the one or more phosphatides or salts thereof is selected from the group consisting of phosphatidyl-choline, phosphatidyl-ethanolamine, phosphatidyl-inositol, and combinations thereof.
